SEQUENCE LISTING (4 sequences)

SEQ ID NO: 1 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 2 - protein, 586 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKG

SEQ ID NO: 3 - protein, 6 aa, Artificial Sequence
  Peptide linker
IEGRMD

SEQ ID NO: 4 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0141296 A1
(43) Pub. Date: May 2, 2024

(54) Compositions and methods for proliferation of mesenchymal stromal cells

(71) Applicant: SWEDISH STROMABIO AB, Solna (SE)

(72) Inventors: Karl-Henrik Grinnemo, Hagersten (SE); Oscar Simonson, Stockholm (SE); Sergey Rodin, Stockholm (SE)

(21) Appl. No.: 18/278,806

(22) PCT Filed: Feb. 24, 2022
(86) PCT No.: PCT/EP2022/054733

§ 371 Date: Aug. 24, 2023

(30) Foreign Application Priority Data
Feb. 25, 2021 (SE) 2150203-4

(51) Int. Cl. C12N 5/0775

(52) U.S. Cl. CPC C12N 5/0662; C12N 2501/599

(57) ABSTRACT

The present invention relates to methods for culturing and isolating cells such as multipotent stem cells or multipotent progenitor cells. The methods of the invention comprise using the human CD105 polypeptide as a cell culture substrate. The cells obtained according to the methods of the invention are capable of forming colony forming unit-fibroblasts (CFU-Fs). Cell lines comprising such cells are useful for the treatment of medical conditions, such as acute respiratory distress syndrome (ARDS).

### TECHNICAL FIELD

The present invention relates to methods for culturing and isolating cells such as multipotent stem cells or multipotent progenitor cells. The methods of the invention comprise using the human CD105 polypeptide as a cell culture substrate. The cells obtained according to the methods of the invention are capable of forming colony forming unit-fibroblasts (CFU-Fs). Cell lines comprising such cells are useful for the treatment of medical conditions, such as acute respiratory distress syndrome (ARDS).

### BACKGROUND ART

Multipotent stem cells are defined by their ability to differentiate into several tissues and to self-renew. The term “mesenchymal stem cells” was earlier attributed to adherent non-hematopoietic fraction of the bone marrow, but later analyses have revealed heterogeneity of the cell population. Individual cells in the adherent fraction of the bone marrow exhibit different levels of multipotency. Therefore, the term “mesenchymal stromal cells” (MSCs), which does not imply homogeneous stemness, has replaced mesenchymal stem cells (Le Blanc and Mougiakakos 2012).

MSCs have been harvested from various tissues and are defined by: (1) expression of certain cell membrane markers (CD73+, CD90+, CD105+); (2) lack of expression of certain markers (CD11b−, CD14−, CD34−, CD45−, CD19−, CD79a−, HLA-DR−); (3) plastic adherence; (4) ability to form colony forming unit-fibroblasts (CFU-Fs); and (5) trilineage multipotency (ability to differentiate into osteoblasts, chondrocytes and adipocytes) in in vitro and in vivo tests (Dominici, Le Blanc et al. 2006). However, there is a growing understanding that these criteria define a very heterogeneous population of cells (Tremain, Korkko et al. 2001) (Bianco, Cao et al. 2013).

Apart from regenerative potential, MSCs have immunoregulatory properties (Uccelli, Moretta et al. 2008). Therefore, MSCs are an attractive source of cell for the regenerative medicine.

Bone marrow derived MSCs (BM-MSCs) have been demonstrated to be safe and potentially effective in different clinical applications (Lalu, McIntyre et al. 2012, von Bahr, Sundberg et al. 2012, Weiss, Casaburi et al. 2013, Weiss 2014). In several preclinical models of acute lung injury MSCs have demonstrated therapeutic potential (Mei, McCarter et al. 2007, Xu, Qu et al. 2008, Lee, Fang et al. 2009, Iyer, Torres-Gonzalez et al. 2010, Danchuk, Ylostalo et al. 2011, Curley, Ansari et al. 2013, Toonkel, Hare et al. 2013, Weiss 2014). While not completely understood, the mechanism of MSCs in these models include release of paracrine anti-inflammatory and anti-bacterial peptides as well as mitochondrial transfer from MSCs into damaged alveolar epithelial cells in the absence of stable MSC engraftment (Islam, Das et al. 2012, Le Blanc and Mougiakakos 2012, Lee, Zhu et al. 2012, Weiss 2014). Another important characteristic of BM-MSCs is their retention in injured lungs after systemic administration (Eggenhofer, Benseler et al. 2012).

Infusion of non-HLA-matched allogeneic MSCs has already been demonstrated to be safe and potentially effective in a widening range of clinical applications, including lung diseases, suggesting that this approach may be beneficial in ARDS (Acute Respiratory Distress Syndrome) patients (Le Blanc, Frassoni et al. 2008, Weiss, Casaburi et al. 2013). A recent phase I dose-escalation safety study demonstrated the safety of a single i.v. administration of 1-10 million cells per kilogram of MSCs in 9 patients with moderate to severe ARDS (Wilson, Liu et al. 2015). A phase II efficacy trial is currently underway, wherein 60 patients with moderate to severe ARDS will be randomized in a 2:1 fashion to BM-MSCs infusion or placebo (Liu, Wilson et al. 2014).

Simonson et al. (Simonson, Mougiakakos et al. 2015) discloses an analysis of the immunomodulatory properties and proteomic profile of MSCs systemically administered to two patients with severe refractory acute respiratory distress syndrome (ARDS). Both patients subsequently improved with resolution of respiratory, hemodynamic, and multiorgan failure. In parallel, a decrease was seen in multiple pulmonary and systemic markers of inflammation, including epithelial apoptosis, alveolar-capillary fluid leakage, and proinflammatory cytokines, microRNAs, and chemokines. In vitro studies of the MSCs demonstrated a broad anti-inflammatory capacity, including suppression of T-cell responses and induction of regulatory phenotypes in T cells, monocytes, and neutrophils.

The efficacy of these therapeutic applications is highly dependent on the heterogeneity of MSC populations (Phinney 2007). Clonal analysis of the existing MSC lines has revealed that single cells differ in the differentiation potential (tri-, bi-, uni- or zero-lineage potent cells) (Russell, Phinney et al. 2010). It has been widely acknowledged that multipotent MSCs are capable of forming CFU-Fs. For MSCs grown under standard conditions, on cell culture treated plastic without additional coating, this ability declines sharply with time (Digirolamo, Stokes et al. 1999) (Madeira, da Silva et al. 2012). Several groups have shown that proliferation of single clones correlates with multipotency (Mareddy, Crawford et al. 2007, Russell, Phinney et al. 2010). Therefore, culturing in vitro on plastic promotes dedifferentiation of multipotent cells rather than provides a growth advantage for subpopulations of cells with lower differentiation potential. There is a need for developing a cell culture system that would promote multipotency of MSCs and facilitate self-renewal of the stem cells.

It has been shown that cell receptor CD105 (SEQ ID NO: 1; also known as Endoglin) is a part of TGF-β receptor complex (Guerrero-Esteo et al. 2002), important for TGF-β signaling (Guerrero-Esteo et al. 2002) and is crucial for angiogenesis (Li et al. 1999). Therefore, CD105 is an important signaling molecule that can bind receptors on the cell membrane of MSCs. Several groups have shown that levels of cell receptor CD105 affect multipotency of MSCs (Mark et al. 2013; Cleary et al. 2016; Anderson et al. 2013).

### BRIEF DESCRIPTION OF THE DRAWING

FIG. 1: Culturing of human MSCs on plates coated with CD105 and control cells grown on uncoated plastic. (A) Growth curves. (B) Results of CFU-Fs assay. * p<0.05. Error bars show standard deviation.

### DESCRIPTION OF THE INVENTION

It is an object of the present invention to overcome the above-identified problems and satisfy the existing needs within the art, i.e. to provide a cell culture system that facilitates self-renewal and promotes multipotency of MSCs.

It has surprisingly been found that culturing of MSCs on plates coated with CD105 (i) enhances proliferation of MSCs, and (ii) supports multipotency and biological activity of MSCs. The inventors have found that activation of MSCs by CD105 provides MSC populations with increased or sustained ability to form CFU-Fs in comparison with that in control cells cultured in standard conditions (on cell culture-treated plastic without any additional coating). This was an unexpected finding, because culturing under standard conditions leads to a sharp decrease in ability to form CFU-Fs.

It is known in the art that the ability to form CFU-Fs correlates with biological activity of MSCs. By providing biologically more active cells, the inventors address an unmet medical need, namely the need for novel therapies capable of treatment of numerous inflammatory diseases.

Consequently, in a first aspect the invention provides a method for culturing multipotent stem cells or multipotent progenitor cells, said method comprising:
(a) coating cell culture plates with a composition comprising a polypeptide selected from the group consisting of:
   (i) a human CD105 polypeptide, said polypeptide comprising the amino acid sequence shown as SEQ ID NO: 1 or SEQ ID NO: 2; and
   (ii) a polypeptide which is a functional variant of a human CD105 polypeptide, said functional variant having at least 80% sequence identity with SEQ ID NO: 1 or SEQ ID NO: 2; and
(b) culturing multipotent stem cells or multipotent progenitor cells on the cell culture plates coated in step (a).

Preferably, the said multipotent stem cell or multipotent progenitor cells are mesenchymal stromal cells (MSCs). The MSCs can be obtained from a source selected from the group consisting of bone marrow, Wharton's jelly, fat tissue, oral cavity, the heart and teeth. Alternatively, the MSCs can be differentiated from stem cells.

In a preferred aspect of the said methods for culturing multipotent stem cells or multipotent progenitor cells, the said composition comprises at least 10% w/w, such as at least 20%, 30%, 40% or 50% w/w of the said polypeptide.

According to the invention, cell culture can take place under various conditions, such as normoxic or hypoxic conditions. Normally, hypoxic conditions for cell culturing are defined as culturing of cells in an atmosphere containing 5% or less of oxygen, while normoxic conditions are defined as culturing of cells in an atmosphere containing from 5% to 21% of oxygen.

In another aspect, the invention provides the use of a composition for culturing multipotent stem cells or multipotent progenitor cells, said composition comprising:
(i) a human CD105 polypeptide, said polypeptide comprising the amino acid sequence shown as SEQ ID NO: 1 or SEQ ID NO: 2; or
(ii) a polypeptide which is a functional variant of a human CD105 polypeptide, said functional variant having at least 80% sequence identity with SEQ ID NO: 1 or SEQ ID NO: 2. Preferably, the said multipotent stem cell or multipotent progenitor cells are mesenchymal stromal cells (MSCs).

In a preferred aspect of the said use for culturing multipotent stem cells or multipotent progenitor cells, the said composition comprises at least 10% w/w, such as at least 20%, 30%, 40% or 50% w/w of the said polypeptide.

Optionally, the said human CD105 polypeptide, or the functional variant thereof, is fused to a portion, such as an Fc portion, of human IgG1. A suitable Fc portion of human IgG1 may comprise the amino acid sequence shown as SEQ ID NO: 4. The IgG1 polypeptide may be connected to the CD105 polypeptide by a peptide linker, such as the linker shown as SEQ ID NO: 3. The CD105-Fc fusion protein is preferably in the form of a homodimer wherein each monomer comprises a human CD105 polypeptide, a linker and an Fc portion of human IgG1. A suitable CD105-Fc fusion protein is commercially available from R&D Systems, Inc. (Catalog No. 6578-EN), and comprises SEQ ID NOS: 2, 3 and 4.

The invention further provides a method for obtaining a multipotent stem cell line or a multipotent progenitor cell line, said method comprising (a) culturing cells using the method of the invention as described above; and (b) isolating a cell line consisting of the cultured cells. The isolation of a cell line can be performed by methods known to the skilled person.

The invention also provides a cell line obtained by this method. Preferably, such a cell line is characterized by a CFU-Fs value (see Experimental Methods, below) which is higher than 5%. The said cell line is useful in medicine, such as in the treatment or prophylaxis of a medical condition selected from the group consisting of: heart insufficiency, heart failure, myocardial infarction, congenital heart disease, myocarditis, valve dysfunction, acute respiratory distress syndrome (ARDS), Critical illness myopathy (CIM), Ventilator induced diaphragm muscle dysfunction (VIDD), graft-versus-host disease (GvHD), solid organ rejections and/or rejections of cell and/or tissue transplants, inflammatory bowel diseases such as Crohn's disease and ulcerative colitis, rheumatoid diseases such as arthritis, any type of inflammation-driven or immunologically induced disease such as multiple sclerosis, ALS, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, and organ failure, such as kidney failure, liver failure, lung failure, or heart failure.

The invention also provides a pharmaceutical composition comprising cells from the cell line obtained by the methods disclosed herein, in combination with at least one pharmaceutically acceptable constituent.

### Definitions

The term “multipotent stem cells” refers to multipotent cells that have an ability to give rise to one or more types of somatic cells (fully differentiated) and have a significant proliferation potential. The term “multipotent progenitor cells” refers to multipotent cells that are direct predecessors of somatic cells. The term “multipotent” means the gene activation potential to differentiate into discrete cell types. Like a stem cell, multipotent progenitor cells specify into particular cell types, however, unlike stem cells, they are the direct predecessors to these cell types and have a limited proliferation potential.

“Mesenchymal stromal cells” or “MSCs” are multipotent stromal cells that are defined by: (1) expression of certain cell membrane markers (CD73+, CD90+, CD105+), (2) lack of expression of certain markers (CD11b−, CD14−, CD34−, CD45−, CD19−, CD79a−, HLA-DR−), (3) plastic adherence, (4) ability to form colony forming-unit fibroblasts (CFU-Fs), and (5) ability to differentiate into a variety of cell types, including osteoblasts (bone cells), chondrocytes (cartilage cells) and adipocytes (fat cells which give rise to marrow adipose tissue) (Dominici, M. et al. Minimal criteria for defining multipotent mesenchymal stromal cells. The International Society for Cellular Therapy position statement. Cytotherapy 8, 315-317 (2006)). MSCs are a mixture of multipotent stem and progenitor cells.

According to the present description and claims, a reference to a product or method “comprising” certain features should be interpreted as meaning that it includes those features, but that it does not exclude the presence of other features, as long as they do not render the invention unworkable. In reference to the compounds or compositions according to the invention, the term “consisting essentially of” means that specific further components can be present, namely those not materially affecting the essential characteristics of the compound or composition.

The term “polypeptide” or “protein” refers to a polymer of the 20 protein amino acids, or amino acid analogs, regardless of its size or function. Thus, exemplary polypeptides include gene products, naturally occurring or native proteins, homologs, orthologs, paralogs, fragments and other equivalents, variants, and analogs of the foregoing.

The term “variant” is used herein to refer to an amino acid sequence that is different from the reference protein by one or more amino acids, e.g., one or more amino acid substitutions, inversions or insertions (additions) or deletions. A variant of a reference protein also refers to a variant of a fragment of the reference protein. A variant can also be a “functional variant,” in which the variant retains some or all of the activity of the reference protein as described herein.

The term “fragment,” when used in reference to a protein, refers to a protein in which amino acid residues are deleted as compared to the reference protein itself, but where the remaining amino acid sequence is usually identical to the corresponding positions in the reference protein. Such deletions can occur at the amino-terminus or carboxy-terminus of the reference protein, or alternatively both. A fragment can also be a “functional fragment,” in which case the fragment retains some or all of the activity of the reference protein as described herein.

Preferably, a functional variant or a functional fragment of a human CD105 polypeptide has at least 60% identity, such as at least 70%, 75%, 80%, 85%, 90%, or 95%, with the amino acid sequence shown as SEQ ID NO: 1 or SEQ ID NO: 2. More preferably, a functional variant or a functional fragment of a human CD105 polypeptide has at least 80% identity with the amino acid sequence shown as SEQ ID NO: 1 or SEQ ID NO: 2.

With regard to the polypeptides and compositions according to the invention, the terms “activity”, and “functional” refer to e.g. one or more of the following features:
(i) supporting cell growth of multipotent stem cells or multipotent progenitor cells;
(ii) increasing or sustaining the ability of multipotent stem cells or multipotent progenitor cells to form CFU-Fs; and/or
(iii) supporting multipotency of multipotent stem cells or multipotent progenitor cells.

In addition, with regard to the polypeptides and compositions according to the invention, the terms “activity”, and “functional” may refer to one or more of the following features:
(iv) sustaining or increasing ability to inhibit proliferation of activated T-cells (for references, see Uccelli, A. et al. (2008) Mesenchymal stem cells in health and disease. Nat Rev Immunol 8: 726-736; and Simonson, O. E. et al. (2015) In Vivo Effects of Mesenchymal Stromal Cells in Two Patients With Severe Acute Respiratory Distress Syndrome. Stem Cells Transl Med 4: 1199-1213);
(v) sustaining or increasing ability to induce regulatory T-cells (see Uccelli et al. and Simonson et al., supra);
(vi) sustaining ability to reduce inflammation in animal models of inflammatory diseases (see Uccelli et al., supra); and/or
(vii) sustaining ability to reduce inflammation in patients with inflammatory diseases (see Uccelli et al., supra).

According to the invention, a phaimaceutical composition may comprise various pharmaceutically acceptable constituents, such as solvents, buffers, carriers, stabilizers, preservatives, etc. The term “pharmaceutically acceptable” means being useful in preparing a pharmaceutical composition that is generally safe, non-toxic and neither biologically nor otherwise undesirable, and includes being useful for veterinary use as well as human pharmaceutical use.

### Experimental Methods

### Cell Culture Substrata

A recombinant human CD105 Fc chimera was purchased from R&D Systems, Inc (Catalog No. 6578-EN). The chimera is a disulfide-linked homodimer wherein each monomer comprises (i) human CD105 (Met1-Gly586; SEQ ID NO: 2); (ii) the peptide linker IEGRMD (SEQ ID NO: 3); and (iii) a human IgG1 Fc portion (SEQ ID NO: 4).

The recombinant human CD105 molecule was expressed in mouse myeloma cell line and purified using Protein A agarose (resin) according to standard methods.

### Cell Culture Dish Coating

CD105 coating: 25 cm² cell culture treated flasks from TPP (Switzerland) were coated overnight at +4° C. with sterile solutions of the recombinant human CD105 chimeric molecule at a concentration of 4.2 μg/ml (0.5 μg/cm²) in phosphate buffered saline (PBS).

Before use, the flasks were incubated at +37° C. for one hour and washed twice with PBS. Prewarmed cell culture medium was then added.

### Culturing of MSCs

BM-MSCs were cultured on cell culture treated flasks with and without the coatings in Dulbecco's Modified Eagle's Medium with low glucose (Life Technologies, California, USA) supplemented with 10% of bovine serum. For passaging, the cells were washed once with phosphate buffered saline (PBS) and removed from the flasks by exposure to TrypLE Express (GIBCO, Thermo Fischer, USA) for approximately 5 minutes. Culturing medium was next added to inhibit TrypLE Express, the cell suspension was centrifuged for 4 minutes at 180×g at room temperature and the supernatant was discarded. After that, the cells were resuspended in prewarmed culture medium, counted and plated at approximately 4000 cells/cm². All cultures were done in humidified cell culture incubators at +37° C. in 5% CO₂.

### Colony Forming Unit-Fibroblasts (CFU-Fs) Test

BM-MSCs were removed from cell culture flasks as described in Culturing of MSCs and counted. Five hundred cells were diluted in 50 ml of the cells culture medium described in Culturing of MSCs, thoroughly mixed and plated on four 10 cm cell culture treated plates (Falcon™), 10 ml of the mixture per plate. Then, the plates were placed in humidified cell culture incubators at +37° C. in 5% CO₂. Five days later, each plate was fed with 5 ml of the cell culture medium. Ten days after the plating, the plates were washed once with PBS, fixed in 4% formaldehyde solution in PBS for 15 minutes at room temperature and washed twice with PBS to remove the residual fixing solution. The colonies were stained by incubation with 0.5% Crystal Violet solution for 10 min, washed twice with water and scored under inverted microscope (Leica, Germany). Cluster of more than 50 cells was counted as one colony.

### Statistics

Statistical significance was determined the by Student's two-tailed t-test for unequal variances.

### EXAMPLES OF THE INVENTION

### Example 1: Mesenchymal Stromal Cells Grown on CD105 Chimeric Molecule Proliferate Faster Than Cells in Standard Conditions and Increase Efficacy in CFU-Fs Test

Bone marrow was aspirated from healthy volunteers after obtaining their informed consent. Bone marrow mononuclear cells were seeded on cell culture treated dished at density of 4000 cells/cm 2 in Dulbecco's Modified Eagle's Medium with low glucose (Life Technologies, California, USA) supplemented with 10% of bovine serum as described above under Experimental Methods (“Culturing of MSC”), passaged three times using standard methods and frozen. FACS analysis showed that the cells were positive for cell membrane markers CD73, CD90, CD105 and negative for markers CD11b, CD14, CD34, CD45, CD19, CD79a, HLA-DR.

The obtained cells were plated at density of 4000 cells/cm² in Dulbecco's Modified Eagle's Medium with low glucose (Life Technologies, California, USA) supplemented with 10% of bovine serum as described above under Experimental Methods (“Culturing of MSC”). The plates were precoated as described above under Experimental Methods (“Cell culture dish coating”) with (a) the CD105 chimeric molecule and (b) no coating (control). The cells were passaged every 7 days. At every passage, the cells were counted and replated at density of 4000 cells/cm².

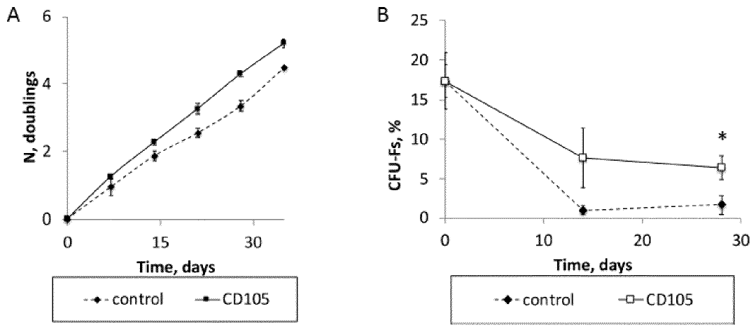

The results show that MSCs cultured on the CD105 chimeric molecule proliferated faster than the cells grown under standard conditions (FIG. 1A).

Every second passage, the clonogenic ability of MSCs was tested in the CFU-Fs assay as described above under Experimental Methods (FIG. 1B). The cells cultured under standard conditions (on plastic) essentially lost their ability to form CFU-Fs after 2 weeks in culture (2 passages during the experiment, 5 in total). Similar results have been reported for MSCs cultured under standard conditions in other labs (Madeira, da Silva et al. 2012).

In contrast, for MSCs grown on the CD105 chimeric molecule, the ability to form colonies was significantly higher (p<0.05) than that of the control cells, even after 1 month in culture (7 passages) (FIG. 1B).

The capability of forming CFU-Fs is a part of the definition of multipotent MSCs (Friedenstein, Chailakhyan et al. 1974) and the efficiency of forming CFU-Fs correlates with multipotency of MSCs (Russell, Phinney et al. 2010). Also, several groups have shown that proliferation rate of single clones, which originate from individualized mesenchymal stromal cells, correlates with multipotency (Mareddy, Crawford et al. 2007) (Russell, Phinney et al. 2010). Therefore, high proliferation rate and sustained ability to form CFU-Fs corroborate with each other and suggest that cells cultured on the CD105 chimeric molecule are more multipotent than cells cultured under standard conditions (control).

### REFERENCES

Anderson P, et al. (2013) “CD105 (Endoglin)-Negative Murine Mesenchymal Stromal Cells Define a New Multipotent Subpopulation with Distinct Differentiation and Immunomodulatory Capacities.” PLOS ONE 8(10): e76979.
Bianco, P., et al. (2013). “The meaning, the sense and the significance: translating the science of mesenchymal stem cells into medicine.” Nature medicine 19(1): 35-42.
Cleary, M.A. et al. (2016). “Expression of CD105 on expanded mesenchymal stem cells does not predict their chondrogenic potential.” Osteoarthritis and Cartilage 24(5):868-872
Curley, G. F., et al. (2013). “Effects of intratracheal mesenchymal stromal cell therapy during recovery and resolution after ventilator-induced lung injury.” Anesthesiology 118(4): 924-932.
Danchuk, S., et al. (2011). “Human multipotent stromal cells attenuate lipopolysaccharide-induced acute lung injury in mice via secretion of tumor necrosis factor-alpha-induced protein 6.” Stem cell research & therapy 2(3): 27.
Digirolamo, C. M., et al. (1999). “Propagation and senescence of human marrow stromal cells in culture: a simple colony-forming assay identifies samples with the greatest potential to propagate and differentiate.” British journal of haematology 107(2): 275-281.
Dominici, M., et al. (2006). “Minimal criteria for defining multipotent mesenchymal stromal cells. The International Society for Cellular Therapy position statement.” Cytotherapy 8(4): 315-317.
Eggenhofer, E., et al. (2012). “Mesenchymal stem cells are short-lived and do not migrate beyond the lungs after intravenous infusion.” Front Immunol 3: 297.
Friedenstein, A. J., et al. (1974). “Stromal cells responsible for transferring the microenvironment of the hemopoietic tissues. Cloning in vitro and retransplantation in vivo.” Transplantation 17(4): 331-340.
Guerrero-Esteo, M. (2002). “Extracellular and Cytoplasmic Domains of Endoglin Interact with the Transforming Growth Factor-β Receptors I and II.” Journal of Biological Chemistry 277(32): 29197-29209.
Islam, M. N., et al. (2012). “Mitochondrial transfer from bone-marrow-derived stromal cells to pulmonary alveoli protects against acute lung injury.” Nat Med 18(5): 759-765.
Iyer, S. S., et al. (2010). “Effect of bone marrow-derived mesenchymal stem cells on endotoxin-induced oxidation of plasma cysteine and glutathione in mice.” Stem cells international 2010: 868076.
Lalu, M. M., et al. (2012). “Safety of cell therapy with mesenchymal stromal cells (SafeCell): a systematic review and meta-analysis of clinical trials.” PloS one 7(10): e47559.
Le Blanc, K., et al. (2008). “Mesenchymal stem cells for treatment of steroid-resistant, severe, acute graft-versus-host disease: a phase II study.” Lancet 371(9624): 1579-1586.
Le Blanc, K. and D. Mougiakakos (2012). “Multipotent mesenchymal stromal cells and the innate immune system.” Nat Rev Immunol 12(5): 383-396.
Le Blanc, K. and D. Mougiakakos (2012). “Multipotent mesenchymal stromal cells and the innate immune system.” Nature reviews. Immunology 12(5): 383-396.
Lee, J. W., et al. (2009). “Allogeneic human mesenchymal stem cells for treatment of E. coli endotoxin-induced acute lung injury in the ex vivo perfused human lung.” Proceedings of the National Academy of Sciences of the United States of America 106(38): 16357-16362.
Lee, J. W., et al. (2012). “Cell-based therapy for acute lung injury: are we there yet?” Anesthesiology 116(6): 1189-1191.
Li D.Y. et al. (1999). “Defective angiogenesis in mice lacking endoglin.” Science 284(5419):1534-1537.
Liu, K. D., et al. (2014). “Design and implementation of the START (STem cells for ARDS Treatment) trial, a phase 1/2 trial of human mesenchymal stem/stromal cells for the treatment of moderate-severe acute respiratory distress syndrome.” Ann Intensive Care 4: 22.
Madeira, A., et al. (2012). “Human mesenchymal stem cell expression program upon extended ex-vivo cultivation, as revealed by 2-DE-based quantitative proteomics.” PloS one 7(8): e43523.
Mareddy, S., et al. (2007). “Clonal isolation and characterization of bone marrow stromal cells from patients with osteoarthritis.” Tissue engineering 13(4): 819-829.
Mark, P. et al. (2013). “Human Mesenchymal Stem Cells Display Reduced Expression of CD105 after Culture in Serum-Free Medium.” Stem Cells International, vol. 2013, Article ID 698076.
Mei, S. H., et al. (2007). “Prevention of LPS-induced acute lung injury in mice by mesenchymal stem cells overexpressing angiopoietin 1.” PLoS medicine 4(9): e269.
Phinney, D. G. (2007). “Biochemical heterogeneity of mesenchymal stem cell populations: clues to their therapeutic efficacy.” Cell cycle 6(23): 2884-2889.
Simonson, O. E., et al. (2015). “In Vivo Effects of Mesenchymal Stromal Cells in Two Patients With Severe Acute Respiratory Distress Syndrome.” Stem Cells Transl Med 4(10): 1199-1213.
Toonkel, R. L., et al. (2013). “Mesenchymal stem cells and idiopathic pulmonary fibrosis. Potential for clinical testing.” American journal of respiratory and critical care medicine 188(2): 133-140.
Tremain, N., et al. (2001). “MicroSAGE analysis of 2,353 expressed genes in a single cell-derived colony of undifferentiated human mesenchymal stem cells reveals mRNAs of multiple cell lineages.” Stem cells 19(5): 408-418.
Uccelli, A., et al. (2008). “Mesenchymal stem cells in health and disease.” Nature reviews. Immunology 8(9): 726-736.
von Bahr, L., et al. (2012). “Long-term complications, immunologic effects, and role of passage for outcome in mesenchymal stromal cell therapy.” Biology of blood and marrow transplantation: journal of the American Society for Blood and Marrow Transplantation 18(4): 557-564.
Weiss, D. J. (2014). “Concise review: current status of stem cells and regenerative medicine in lung biology and diseases.” Stem Cells 32(1): 16-25.
Weiss, D. J., et al. (2013). “A placebo-controlled, randomized trial of mesenchymal stem cells in COPD.” Chest 143(6): 1590-1598.
Wilson, J. G., et al. (2015). “Mesenchymal stem (stromal) cells for treatment of ARDS: a phase 1 clinical trial.” Lancet Respir Med 3(1): 24-32.
Xu, J., et al. (2008). “Mesenchymal stem cell-based angiopoietin-1 gene therapy for acute lung injury induced by lipopolysaccharide in mice.” The Journal of pathology 214(4): 472-481.

```
Met Asp Arg Gly Thr Leu Pro Leu Ala Val Ala Leu
1 5 10
Leu Leu Ala Ser Cys Ser Leu Ser Pro Thr Ser Leu
                15 20
Ala Glu Thr Val His Cys Asp Leu Gln Pro Val Gly
25 30 35
Pro Glu Arg Gly Glu Val Thr Tyr Thr Thr Ser Gln
                        40 45
Val Ser Lys Gly Cys Val Ala Gln Ala Pro Asn Ala
        50 55 60
Ile Leu Glu Val His Val Leu Phe Leu Glu Phe Pro
                                65 70
Thr Gly Pro Ser Gln Leu Glu Leu Thr Leu Gln Ala
                75 80
Ser Lys Gln Asn Gly Thr Trp Pro Arg Glu Val Leu
85 90 95
Leu Val Leu Ser Val Asn Ser Ser Val Phe Leu His
                        100 105
Leu Gln Ala Leu Gly Ile Pro Leu His Leu Ala Tyr
        110 115 120
Asn Ser Ser Leu Val Thr Phe Gln Glu Pro Pro Gly
                                125 130
Val Asn Thr Thr Glu Leu Pro Ser Phe Pro Lys Thr
                135 140
Gln Ile Leu Glu Trp Ala Ala Glu Arg Gly Pro Ile
145 150 155
Thr Ser Ala Ala Glu Leu Asn Asp Pro Gln Ser Ile
                        160 165
Leu Leu Arg Leu Gly Gln Ala Gln Gly Ser Leu Ser
        170 175 180
Phe Cys Met Leu Glu Ala Ser Gln Asp Met Gly Arg
                                185 190
Thr Leu Glu Trp Arg Pro Arg Thr Pro Ala Leu Val
                195 200
Arg Gly Cys His Leu Glu Gly Val Ala Gly His Lys
205 210 215
Glu Ala His Ile Leu Arg Val Leu Pro Gly His Ser
                        220 225
Ala Gly Pro Arg Thr Val Thr Val Lys Val Glu Leu
        230 235 240
Ser Cys Ala Pro Gly Asp Leu Asp Ala Val Leu Ile
                                245 250
Leu Gln Gly Pro Pro Tyr Val Ser Trp Leu Ile Asp
                255 260
Ala Asn His Asn Met Gln Ile Trp Thr Thr Gly Glu
265 270 275
Tyr Ser Phe Lys Ile Phe Pro Glu Lys Asn Ile Arg
                        280 285
Gly Phe Lys Leu Pro Asp Thr Pro Gln Gly Leu Leu
        290 295 300
Gly Glu Ala Arg Met Leu Asn Ala Ser Ile Val Ala
                                305 310
Ser Phe Val Glu Leu Pro Leu Ala Ser Ile Val Ser
                315 320
Leu His Ala Ser Ser Cys Gly Gly Arg Leu Gln Thr
325 330 335
Ser Pro Ala Pro Ile Gln Thr Thr Pro Pro Lys Asp
                        340 345
Thr Cys Ser Pro Glu Leu Leu Met Ser Leu Ile Gln
        350 355 360
Thr Lys Cys Ala Asp Asp Ala Met Thr Leu Val Leu
                                365 370
Lys Lys Glu Leu Val Ala His Leu Lys Cys Thr Ile
                375 380
Thr Gly Leu Thr Phe Trp Asp Pro Ser Cys Glu Ala
385 390 395
Glu Asp Arg Gly Asp Lys Phe Val Leu Arg Ser Ala
                        400 405
Tyr Ser Ser Cys Gly Met Gln Val Ser Ala Ser Met
        410 415 420
Ile Ser Asn Glu Ala Val Val Asn Ile Leu Ser Ser
                                425 430
Ser Ser Pro Gln Arg Lys Lys Val His Cys Leu Asn
                435 440
Met Asp Ser Leu Ser Phe Gln Leu Gly Leu Tyr Leu
445 450 455
Ser Pro His Phe Leu Gln Ala Ser Asn Thr Ile Glu
                        460 465
Pro Gly Gln Gln Ser Phe Val Gln Val Arg Val Ser
        470 475 480
Pro Ser Val Ser Glu Phe Leu Leu Gln Leu Asp Ser
                                485 490
Cys His Leu Asp Leu Gly Pro Glu Gly Gly Thr Val
                495 500
Glu Leu Ile Gln Gly Arg Ala Ala Lys Gly Asn Cys
505 510 515
Val Ser Leu Leu Ser Pro Ser Pro Glu Gly Asp Pro
                        520 525
Arg Phe Ser Phe Leu Leu His Phe Tyr Thr Val Pro
        530 535 540
Ile Pro Lys Thr Gly Thr Leu Ser Cys Thr Val Ala
                                545 550
Leu Arg Pro Lys Thr Gly Ser Gln Asp Gln Glu Val
                555 560
His Arg Thr Val Phe Met Arg Leu Asn Ile Ile Ser
565 570 575
Pro Asp Leu Ser Gly Cys Thr Ser Lys Gly Leu Val
                        580 585
Leu Pro Ala Val Leu Gly Ile Thr Phe Gly Ala Phe
        590 595 600
Leu Ile Gly Ala Leu Leu Thr Ala Ala Leu Trp Tyr
                                605 610
Ile Tyr Ser His Thr Arg Ser Pro Ser Lys Arg Glu
                615 620
Pro Val Val Ala Val Ala Ala Pro Ala Ser Ser Glu
625 630 635
Ser Ser Ser Thr Asn His Ser Ile Gly Ser Thr Gln
                        640 645
Ser Thr Pro Cys Ser Thr Ser Ser
        650 655
```

1. A method for culturing multipotent stem cells or multipotent progenitor cells, said method comprising
(a) coating cell culture plates with a composition comprising a polypeptide selected from the group consisting of:
(i) a human CD105 polypeptide, said polypeptide comprising the amino acid sequence shown as SEQ ID NO: 1 or SEQ ID NO: 2; and
(ii) a polypeptide which is a functional variant of a human CD105 polypeptide, said functional variant having at least 80% sequence identity with SEQ ID NO: 1 or SEQ ID NO: 2; and
(b) culturing multipotent stem cells or multipotent progenitor cells, on the cell culture plates coated in step (a).

2. The method according to claim 1 wherein the said multipotent stem cells or multipotent progenitor cells are mesenchymal stromal cells (MSCs).

3. The method according to claim 1 or 2, wherein the composition comprises at least 10% w/w of the polypeptide.

4. The method according to claim 2 wherein the MSCs are obtained from a source selected from the group consisting of bone marrow, Wharton's jelly, fat tissue, oral cavity, the heart, and teeth.

5. The method according to claim 2 wherein the MSCs are differentiated from stem cells.

6. Use of a composition for culturing multipotent stem cells or multipotent progenitor cells, said composition comprising:
(i) a human CD105 polypeptide, said polypeptide comprising the amino acid sequence shown as SEQ ID NO: 1 or SEQ ID NO: 2; or
(ii) a polypeptide which is a functional variant of a human CD105 polypeptide, said functional variant having at least 80% sequence identity with SEQ ID NO:
1 or SEQ ID NO: 2.

7. The use according to claim 6 wherein the said multipotent stem cells or multipotent progenitor cells are mesenchymal stromal cells (MSCs).

8. The use according to claim 6 or 7, wherein the composition comprises at least 10% w/w of the polypeptide.

9. A method for obtaining a multipotent stem cell line or multipotent progenitor cell line, comprising:
(a) culturing cells by the method according to any one of claims 1 to 5; and
(b) isolating a cell line consisting of the cultured multipotent stem or progenitor cells.

10. A cell line obtained by the method according to claim 9.

11. The cell line according to claim 10, wherein the cell line is characterized by a CFU-Fs value higher than 5%.

12. The cell line according to claim 10 or 11 for use in medicine.

13. The cell line according to claim 10 or 11 for use in the treatment or prophylaxis of a medical condition selected from the group consisting of: heart insufficiency, heart failure, myocardial infarction, congenital heart disease, myocarditis, valve dysfunction, acute respiratory distress syndrome (ARDS), Critical illness myopathy (CIM), Ventilator induced diaphragm muscle dysfunction (VIDD), graft-versus-host disease (GvHD), solid organ rejections and/or rejections of cell and/or tissue transplants, inflammatory bowel diseases such as Crohn's disease and ulcerative colitis, rheumatoid diseases such as arthritis, any type of inflammation-driven or immunologically induced disease such as multiple sclerosis, ALS, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, and organ failure, such as kidney failure, liver failure, lung failure, or heart failure.

14. The cell line according for use according to claim 13, wherein the medical condition is acute respiratory distress syndrome (ARDS).

15. A pharmaceutical composition comprising cells from the cell line according to claim 10 or 11, in combination with at least one pharmaceutically acceptable constituent.
